(19) United States
(12) Patent Application Publication
(10) Pub. No.: US 2026/0266705 A1
(43) Pub. Date: Sep. 10, 2026

(54) LARGE-SCALE INDOOR SIMULATION DEVICE AND METHOD FOR IN-SITU SOIL LAYERS

(71) Applicant: China Institute of Water Resources and Hydropower Research, Beijing (CN)

(72) Inventors: Zhengquan YANG, Beijing (CN); Yiying ZHAO, Beijing (CN); Xiaosheng LIU, Beijing (CN); Jianming ZHAO, Beijing (CN); Shaokun WANG, Beijing (CN); Kaibin ZHU, Beijing (CN); Qiwang LIU, Beijing (CN); Shuanwang ZHANG, Beijing (CN); Xiangqian LIANG, Beijing (CN); Long WANG, Beijing (CN); Jingjun LI, Beijing (CN); Zhongyang YU, Beijing (CN); Rongping YU, Beijing (CN); Qiao YU, Beijing (CN); Hongsen MA, Beijing (CN)

(73) Assignee: China Institute of Water Resources and Hydropower Research, Beijing (CN)

(21) Appl. No.: 19/558,349

(22) Filed: Mar. 5, 2026

(30) Foreign Application Priority Data
Mar. 6, 2025 (CN) 202510263963.9

(51) Int. Cl. G01N 3/08; G01N 33/24

(52) U.S. Cl. CPC G01N 3/08; G01N 33/24; G01N 2203/0019; G01N 2203/0085

(57) ABSTRACT

A large-scale indoor simulation device includes: a base bottom plate, where the base bottom plate is connected, through a cooperative loading jack, to a material barrel having a measurement hole, an inner wall of the material barrel is provided with a flexible sleeve, and an open end of the material barrel is provided with an upper cover plate having a variable-diameter test hole; a support column is utilized to penetrate the upper cover plate and the base bottom plate and is fixed by a nut; the cooperative loading jack is internally provided with a force sensor, an outer side of the cooperative loading jack is provided with a displacement sensor, and the cooperative loading jack, the force sensor, and the displacement sensor are all connected to a loading console.

The present application claims priority to Chinese Patent Application No. 202510263963.9 filed on March 6, 2025, entitled “Large-Scale Indoor Simulation Device and Method for In-Situ Soil Layers”, the content of which is incorporated herein by reference in its entirety.

### TECHNICAL FIELD

The present application relates to the field of indoor soil testing equipment, and in particular to a large-scale indoor simulation device and method for in-situ soil layers.

### BACKGROUND

The statements in this section merely provide background technical information related to the present application and do not necessarily constitute prior art.

Currently, the static and dynamic characteristics of soil are studied, and corresponding characteristic parameters are determined, primarily by conducting indoor experiments on reconstituted soil materials. Indoor static and dynamic characteristic experiments may simulate external control conditions such as various stress conditions of the soil, and obtain various types of static and dynamic constitutive model characteristic parameters of the soil through systematic tests. However, it is difficult to reflect the in-situ structural characteristics of natural soil layers, and it is difficult to reliably determine the characteristic parameters of the soil solely by indoor experiments. Field in-situ experiments are conducted at actual soil layers, with small disturbance to the in-situ soil, which may more truly reflect the in-situ structural effects and in-situ stress states of the soil, and may also solve the size effect problems caused by equipment size limitations in indoor experiments. However, the experiment conditions of field in-situ experiments are relatively few and difficult to regulate; the experiment stress conditions are few, and it is difficult to conduct experiments under different consolidation stress states and difficult to verify the complex influence relationships of various factors such as in-situ compactness (relative density).

Simulating field in-situ conditions indoors, constructing soil layers that may reflect the actual field state, and conducting calibration experiments of field in-situ tests under controllable conditions are reasonable approaches to solve problems in the existing single indoor reconstituted soil material experiments and field in-situ experiments, and to integrate the advantages of both to comprehensively study and determine the static and dynamic characteristic parameters of the overburden soil.

### SUMMARY

To solve the problems in the above background, the present application provides a large-scale indoor simulation device and method for in-situ soil layers. The present application develops the large-scale indoor simulation device for in-situ soil layers and proposes a corresponding experiment method for conducting calibration experiments of indoor simulated field in-situ tests, which may extend the few and certain field experiment conditions to different in-situ conditions, and provides necessary equipment and technical support for reliably determining mechanical characteristics and parameters of the in-situ soil layers by combining results of the field in-situ experiment and indoor experiment.

For above objectives, the present application adopts the following technical solutions.

The present application provides a large-scale indoor simulation device for in-situ soil layers.

A large-scale indoor simulation device for in-situ soil layers, including: a base bottom plate, where the base bottom plate is connected, through a cooperative loading jack, to a material barrel having a measurement hole, an inner wall of the material barrel is provided with a flexible sleeve, and an open end of the material barrel is provided with an upper cover plate having a variable-diameter test hole; a support column is utilized to penetrate the upper cover plate and the base bottom plate and is fixed by a nut; the cooperative loading jack is internally provided with a force sensor, an outer side of the cooperative loading jack is provided with a displacement sensor, and the cooperative loading jack, the force sensor, and the displacement sensor are all connected to a loading console.

Further, the support column is provided with a directional bearing, the directional bearing includes a bearing bracket and a bearing, the support column passes through the bearing bracket, the directional bearing is fastened to the support column by utilizing the bearing bracket, and the bearing is in vertical contact with an outer side wall of the material barrel.

Further, a bolt having a variable-diameter hole is provided in the upper cover plate, a variable-diameter hole inner bolt is provided in the bolt having the variable-diameter hole, and a center of the variable-diameter hole inner bolt, a center of the bolt having the variable-diameter hole, and a center of the material barrel are coaxial.

Further, a positioning nut is utilized to fix between the support column and the upper cover plate, and a fastening nut is utilized to fix between the support column and the base bottom plate.

Further, a hole is provided at the flexible sleeve at a position corresponding to the measurement hole of the material barrel.

Further, a plurality sets of measurement hole bolts are provided at a side wall of the material barrel.

Further, a lubricating material is applied between the flexible sleeve and the material barrel.

The present application provides a large-scale indoor simulation method for in-situ soil layers.

A large-scale indoor simulation method for in-situ soil layers, applied to the large-scale indoor simulation device described above, including:

placing a thin-layer flexible sleeve having an outer wall coated with a lubricating material (such as Vaseline or carbon powder) into the material barrel, to closely fit with the inner wall of the material barrel; providing a hole at a position of the flexible sleeve corresponding to a position of the measurement hole of the material barrel;

loading experiment soil materials in layers into an interior of the material barrel having the measurement hole according to a preset relative density, and performing compaction for compactness restoration by utilizing a compactor after each layer is loaded; after a round of compaction, measuring and comparing whether the experiment soil materials are compacted to a designated height, and if not, performing a next round of compaction until the designated height is reached to complete loading and compaction of a layer of the experiment soil materials; during the compaction, removing a corresponding measurement hole bolt and arranging a sensor at a required position according to an experiment requirement;

installing the upper cover plate, starting the cooperative loading jack through the loading console, and loading the experiment soil materials to a preset stress state according to a signal of the force sensor to complete loading; and

conducting an indoor simulation experiment of a field in-situ test.

Further, the compaction separately adopts manual compaction and electric compaction according to a soil sample density requirement; during the manual compaction or the electric compaction, the compaction follows a basic principle of compacting from outside to inside, tamping a circle along the inner wall of the material barrel having the measurement hole, and moving the compactor inward to perform a next circle of compaction along an inner diameter of a compaction trajectory; during the compaction, two compaction trajectories partially overlap to ensure that a compaction range completely covers the experiment soil materials.

Further, a variable-diameter hole inner bolt or a bolt having a variable-diameter hole is removed before conducting the indoor simulation experiment.

Compared with the prior art, the beneficial effects of the present application are as follows.

To reliably determine mechanical characteristics and parameters of soil by combining results of the field in-situ experiment and indoor experiment, it is necessary to conduct calibration experiments of indoor simulated field in-situ tests. The present application develops a large-scale indoor simulation device for in-situ soil layers and proposes a corresponding experiment method. A rigid side wall is utilized to restrict lateral deformation of the soil, and a vertical load is applied through a jack to simulate an in-situ stress state of the in-situ soil layers; an in-situ density of the in-situ soil layers is simulated by controlling a sample preparation method. The device provides variable-diameter test holes and a plurality of measurement holes, which provides basic conditions for further conducting indoor simulation experiments of various field tests. The present application may simulate in-situ states such as density and stress of the in-situ soil layers in indoor simulated field tests, providing necessary equipment and technical support for conducting calibration experiments of indoor simulated field in-situ tests.

The present application has a flexible sleeve placed inside, the flexible sleeve is in smooth contact with the inner wall of the material barrel, and completely deforms coordinately with soil materials during the experiment, achieving an objective of reducing friction between soil material and the wall of the material barrel, and keeping a stress state inside soil materials as consistent as possible.

### BRIEF DESCRIPTION OF THE DRAWINGS

The drawings of the specification constituting a part of the present application are used to provide a further understanding of the present application, and the schematic embodiments of the present application and the description thereof are used to explain the present application and do not constitute an improper limitation of the present application.

FIG. 1 is a structural diagram of a large-scale indoor simulation device for in-situ soil layers shown in the present application.

FIG. 2 is a detail mechanism diagram of an upper cover plate having a variable-diameter test hole shown in the present application.

FIG. 3 is a detail mechanism diagram of a base bottom plate shown in the present application.

FIG. 4 is a detail mechanism diagram of a directional bearing shown in the present application.

### DETAILED DESCRIPTION OF THE EMBODIMENTS

The present application is further described below in conjunction with the drawings and embodiments.

It should be pointed out that the following detailed description is exemplary and is intended to provide a further description of the present application. Unless otherwise specified, all technical and scientific terms used herein have the same meaning as commonly understood by a person of ordinary skill in the technical field to which the present application belongs.

It should be noted that the terms used herein are merely for describing implementations and are not intended to limit exemplary embodiments according to the present application. As used herein, unless the context clearly indicates otherwise, the singular forms are also intended to include the plural forms. In addition, it should also be understood that when the terms “comprise” and/or “include” are used in this specification, they indicate the presence of features, steps, operations, devices, components, and/or combinations thereof.

The in-situ structure of the in-situ soil layers is extremely easy to be destroyed due to sampling disturbance during sampling. The field large-scale special sampling method is not applicable to general engineering due to high costs. Results of indoor reconstituted sample experiments are difficult to reflect the influence of the in-situ structure and difficult to accurately reflect the physical and mechanical properties of the soil. Experiment conditions of field in-situ experiments are few and difficult to regulate, and characteristic parameters of static and dynamic constitutive models of the soil may not be directly obtained. Therefore, it is necessary to conduct calibration experiments of indoor simulated field in-situ tests, extending the few and certain field experiment conditions to different in-situ conditions, and providing necessary conditions for reliably determining mechanical characteristics and parameters of the in-situ soil layers by combining results of the field in-situ experiment and indoor reconstituted sample experiment. Existing indoor experiment devices are all dedicated experiment equipment for specific experiments, lacking experiment devices and technologies that may restore the state of in-situ soil and provide basic conditions for conducting various field test simulation experiments, which restricts the development of indoor simulation experiment research of field in-situ tests. To give full play to the respective advantages of indoor and outdoor experiments, and to reliably determine the mechanical characteristics and parameters of the in-situ soil layers by combining results of the field in-situ experiment and indoor experiment, it is necessary to conduct calibration experiments of indoor simulated field in-situ tests and extend few and certain field experiment conditions to different in-situ conditions. It is necessary to develop a large-scale indoor simulation device and method for in-situ soil layers that may restore the state of field soil layers. The present application is described in detail below through several embodiments.

### Embodiment I

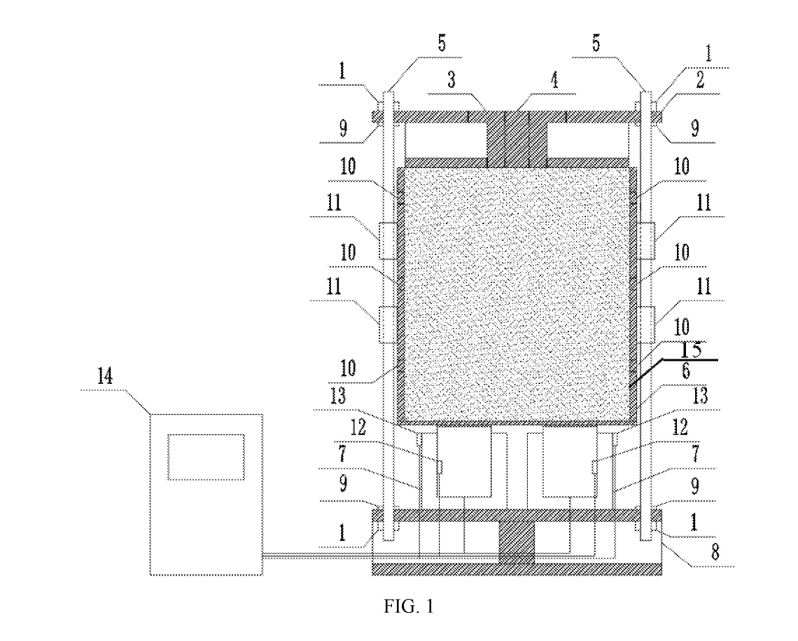

As shown in FIG. 1, this embodiment provides a large-scale indoor simulation device for in-situ soil layers. Components in the figure are respectively: fastening nut 1; upper cover plate 2 having a variable-diameter test hole; bolt 3 having a variable-diameter hole; variable-diameter hole inner bolt 4; support column 5; material barrel 6 having a measurement hole; cooperative loading jack 7; base bottom plate 8; positioning nut 9; measurement hole bolt 10; directional bearing 11; force sensor 12; displacement sensor 13; loading console 14; and flexible sleeve 15.

A test hole diameter of the upper cover plate 2 is determined according to experiment requirements of a hole diameter in field in-situ tests for which indoor simulation experiments are to be conducted, and a material stiffness of the upper cover plate 2 should satisfy deformation requirements during loading; a measurement hole diameter of the material barrel 6 is determined according to installation size requirements of a sensor to be arranged, and a barrel diameter and a height of the material barrel 6 are determined according to a maximum particle size of soil materials and satisfy requirements of the field in-situ tests for which indoor simulation experiments are to be conducted; a stiffness of a side wall material of the material barrel 6 should be far greater than a soil stiffness to satisfy lateral deformation requirements during loading, and steel may be adopted.

The present application determines dimensions of the material barrel 6 through a maximum particle size of the soil materials, and the central soil is minimally affected by side wall constraints during loading and experiment, and boundary effects may be ignored.

The side wall of the material barrel 6 of the present application is a rigid boundary with a stiffness far greater than the soil stiffness, which may strictly control its own lateral deformation. Ascending and descending of the cooperative loading jack are controlled through the loading console, and a vertical load is applied to soil samples by the upper cover plate providing a reaction force, which restores the soil stress state of the in-situ soil layers.

A thin-layer flexible sleeve 15 is provided in the material barrel 6, and the flexible sleeve 15 is closely fitted with the inner wall of the material barrel 6. A lubricating material (such as Vaseline, carbon powder, etc.) is applied to an outer wall of the flexible sleeve 15 to reduce friction with the material barrel 6. An inner wall of the flexible sleeve 15 is in rough contact with soil materials, and the flexible sleeve 15 completely deforms coordinately with soil materials during the experiment, without affecting deformation characteristics of soil materials during the experiment. This method may reduce friction between soil materials and the wall of the material barrel and keep stress states of soil materials inside the material barrel as consistent as possible.

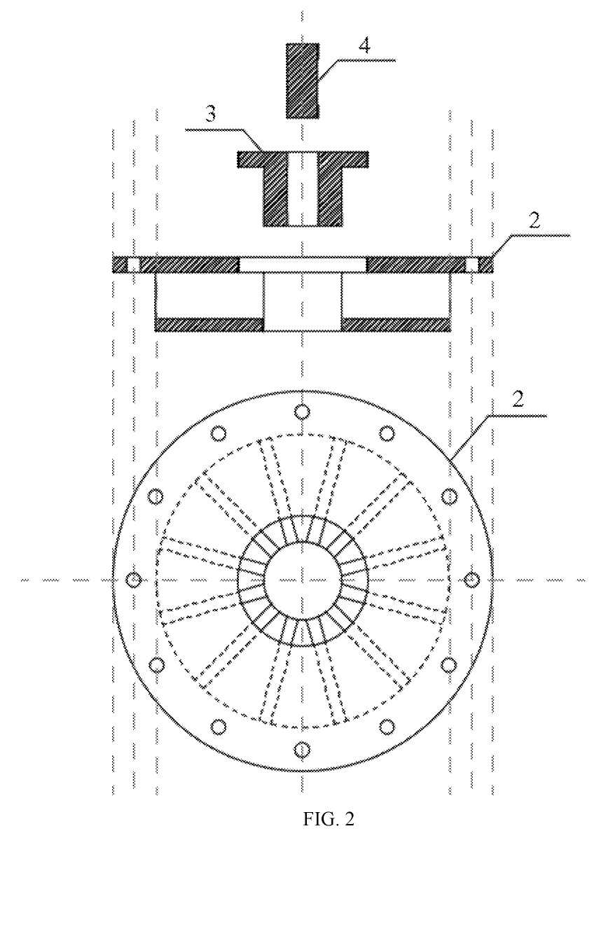

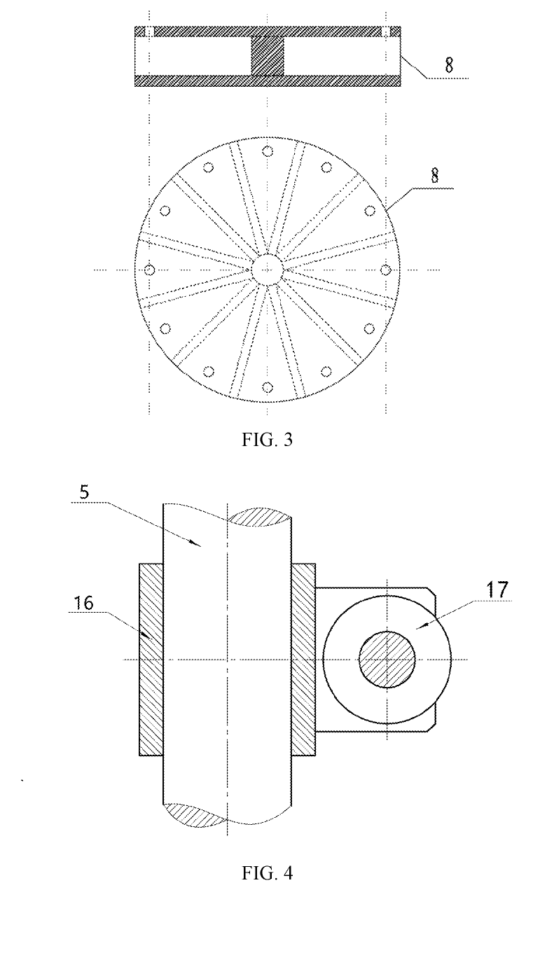

Detail mechanisms of the upper cover plate 2 and the base bottom plate 8 of the large-scale indoor simulation device for in-situ soil layers are respectively shown in FIG. 2 and FIG. 3. Different field in-situ experiments have different requirements for experiment hole diameters. To satisfy indoor simulation requirements of various in-situ experiments, a variable-diameter test hole is designed in the upper cover plate, and a plurality sets of replaceable bolts 3 having a variable-diameter hole and corresponding variable-diameter hole inner bolts 4 are utilized to satisfy experiment requirements. According to experiment hole diameters required for different experiments, a bolt 3 having a variable-diameter hole and a corresponding variable-diameter hole inner bolt 4 matching the diameter may be selected for installation before a loading stage. During an experiment, the variable-diameter hole inner bolt 4 may be separately removed or the bolt 3 having the variable-diameter hole together with the variable-diameter hole inner bolt 4 may be removed as needed, leaving an experiment hole of a size required by the experiment for experiments of in-situ tests. This method, on a basis of satisfying experiment hole requirements, maximally avoids unloading of a load above the soils except at a position of the experiment hole, avoiding affecting accuracy of subsequent experiment results.

The present application adopts the replaceable bolt 3 having the variable-diameter hole and variable-diameter hole inner bolt 4, which may satisfy requirements of indoor simulation experiments for various in-situ tests. In the present application, shapes, sizes, and quantities of the bolt 3 having the variable-diameter hole, the variable-diameter hole inner bolt 4, and the measurement hole bolt 10 may be changed according to actual working condition requirements, such as utilizing a multi-layer bolt having a hole. The present application is not limited thereto. The present application may change geometric forms of rib plates of components or use solid components instead.

Detail mechanisms of the directional bearing 11 of the large-scale indoor simulation device for in-situ soil layers are shown in FIG. 4. The directional bearing 11 is composed of a bearing bracket 16 and a bearing 17. During use, the support column 5 passes through the bearing bracket 16, and the directional bearing 11 is fastened to the column 5 by utilizing the bearing bracket 16. Each directional bearing 17 is in vertical contact with the material barrel 6 to ensure that the material barrel 6 does not deviate in that direction. Meanwhile, friction between the bearing 17 and the material barrel 6 is reduced as much as possible during loading and unloading stages of the device, avoiding influence of vertical friction on experiment results.

In the present application, other directional devices may also be used to replace the directional bearing, and the present application is not limited thereto.

A loading device for providing an in-situ stress state for the large-scale indoor simulation device for in-situ soil layers includes a loading console 14, a cooperative loading jack 7, a force sensor 12, and a displacement sensor 13. After numerical values of a target force and a target displacement are set on an operation interface of the loading console 14, the loading console 14 compares the target force and the target displacement with a force and a displacement measured by the force sensor 12 and the displacement sensor 13, and ascending or descending of the cooperative loading jack 7 below the material barrel 6 having the measurement hole may be automatically controlled through the loading console 14, driving the material barrel 6 having the measurement hole to ascend or descend; when the target force or the target displacement set in the loading console 14 is equal to the force or the displacement measured by the force sensor 12 and the displacement sensor 13, the cooperative loading jack 7 stops ascending or descending, and a position of the cooperative loading jack 7 remains unchanged. During loading and unloading, a reaction force is provided by the upper cover plate 2 having the variable-diameter test hole and the bolt 3 having the variable-diameter hole and the variable-diameter hole inner bolt 4 in the upper cover plate 2 having the variable-diameter test hole to apply a vertical load to soil samples, simulating the in-situ stress state of soil layers.

During loading and unloading, a plurality of cooperative loading jacks 7 uniformly arranged below the material barrel 6 having the measurement hole perform cooperative movement based on a displacement signal provided by the displacement sensor 13, to ensure that a bottom surface of the material barrel 6 having the measurement hole remains horizontal, and a movement direction of the material barrel 6 having the measurement hole always remains vertical.

The present application adopts a loading mode of coordinated loading by a plurality of jacks, which may ensure that the movement direction of the material barrel always remains vertical, effectively reducing a phenomenon of uneven stress distribution of soil layers during loading and experiments. In the present application, the quantity of jacks may be changed according to working conditions and requirements, such as adopting a single jack, or utilizing other loading device instead of the jack. The present application is not limited thereto.

An installation flow of the large-scale indoor simulation device for in-situ soil layers is as follows.

(1) The base bottom plate 8 is placed on a site where flatness and stiffness satisfy requirements.

(2) The force sensor 12 is installed into the cooperative loading jack 7. The cooperative loading jack 7 is placed on the base bottom plate 8 to ensure that a top surface of the cooperative loading jack 7 is horizontal and at a consistent height.

(3) The material barrel 6 having the measurement hole is placed on the top surface of the cooperative loading jack 7, and a center position of the material barrel 6 having the measurement hole remains consistent with a center position of the base bottom plate 8.

(4) The support column 5 passes through a lower positioning nut 9 and the base bottom plate 8 in sequence, and a lower fastening nut 1 is installed and tightened; the directional bearing 11 and an upper positioning nut 9 are installed at the support column 5 in sequence. The present application installs the directional bearing 11 at the support column 5, which may effectively prevent the device from tilting during installation, loading, and experiments.

(5) The support column 5 passes through the upper cover plate 2 having the variable-diameter test hole, and the upper cover plate 2 having the variable-diameter test hole is placed on the upper positioning nut 9; after confirming that a center position of the upper cover plate 2 having the variable-diameter test hole is consistent with the center position of the base bottom plate 8, the fastening nut 1 at an upper part of the support column 5 is tightened.

(6) The variable-diameter hole inner bolt 4 is installed into the bolt 3 having the variable-diameter hole, and then the bolt 3 having the variable-diameter hole is installed into the upper cover plate 2 having the variable-diameter test hole.

(7) Measurement hole bolts 10 are tightened at the side wall of the material barrel 6 having the measurement hole. The side wall of the material barrel 6 of the present application is provided with a plurality sets of symmetrical measurement hole bolts 10, and corresponding sensors may be installed and arranged through measurement holes according to experiment requirements.

(8) The displacement sensor 13 is installed to an outer side of the cooperative loading jack 7.

(9) The loading console 14 is connected to the cooperative loading jack 7, and then the force sensor 12 and the displacement sensor 13 are connected to the loading console 14.

(10) The loading condition of the cooperative loading jack 7 and signals of the force sensor 12 and the displacement sensor 13 are tested through the loading console 14 to complete the installation.

The present application develops a large-scale indoor simulation device for in-situ soil layers and proposed a corresponding experiment method, which may achieve the in-situ states such as density and stress of in-situ soil layers in indoor simulated field tests; meanwhile, the variable-diameter test hole and a plurality of measurement holes are provided, providing basic conditions for further conducting indoor simulation experiments of various field tests.

The present application achieves precise control of soil sample compactness through a sample preparation method combining layered sample preparation, manual compaction, and electric compaction, which may restore various soil compactness states from loose to dense.

### Embodiment II

This embodiment provides a large-scale indoor simulation method for field in-situ soil layers, applied to the large-scale indoor simulation device for in-situ soil layers according to Embodiment I, including the following.

The upper fastening nut 1 is taken off, and the upper cover plate 2 having the variable-diameter test hole is removed. The thin-layer flexible sleeve 15 with the outer wall coated with a lubricating material (such as Vaseline, carbon powder, etc.) is placed into the material barrel 6, to closely fit with the inner wall of the material barrel 6; a hole is provided at a position of the flexible sleeve corresponding to a position of the measurement hole of the material barrel 6.

Experiment soil materials are loaded in layers into an interior of the material barrel 6 having the measurement hole according to a preset relative density. After each layer is loaded, compaction is performed by utilizing a compactor, supplemented by water spraying if necessary, to perform in-situ soil layer compactness restoration. Manual compaction or electric compaction is separately adopted according to soil sample density requirements. Preparation of relatively loose soil samples adopts manual compaction, utilizing a metal compactor to perform tamping on the soils. Preparation of relatively dense soil samples adopts electric compaction, utilizing an electric mechanical compactor to perform tamping on the soils. During manual compaction or utilizing a small electric mechanical compactor, compaction follows a basic principle of compacting from outside to inside: first tamping a circle along the inner wall of the material barrel 6 having the measurement hole, then moving the compactor inward to perform a next circle of compaction along an inner diameter of the compaction trajectory; it is noted that two compaction trajectories should have an overlap of approximately 20% to ensure that a compaction range completely covers the experiment soil materials. The above compaction is repeated until compaction reaches a center of the material barrel 6 having the measurement hole, completing a round of compaction. Whether compaction has reached a designated height is measured and compared; if not, the above steps are repeated to perform a next round of compaction until the designated height is reached, completing loading and compaction of a layer of soil materials. During the compaction, according to experiment requirements, corresponding measurement hole bolts 10 are removed at a required position and sensors are arranged.

After loading and compaction of the soil materials in the material barrel 6 having the measurement hole are completed, the upper cover plate 2 having the variable-diameter test hole is installed by aligning with the support column 5 according to a hole position, and the upper fastening nut 1 is tightened.

A suitable bolt 3 having a variable-diameter hole and a variable-diameter hole inner bolt 4 are selected according to experiment requirements and installed at the upper cover plate 2 having the variable-diameter test hole.

The cooperative loading jack 7 is started through the loading console 14. Based on a signal of the force sensor 12, the soil materials are loaded to a preset stress state to complete loading.

The variable-diameter hole inner bolt 4 or the bolt 3 having the variable-diameter hole is removed as needed to conduct indoor simulation experiments of field in-situ tests. After removing the variable-diameter hole inner bolt 4 or the bolt 3 having the variable-diameter hole, field in-situ test equipment is assembled and erected on the upper cover plate 2 having the variable-diameter test hole, and experiment soil materials of the indoor simulation experiments of field in-situ tests are located at a center of the variable-diameter hole inner bolt 4 or the bolt 3 having the variable-diameter hole; simulation experiments are conducted according to experiment operation requirements of the field in-situ tests, and experiment data is recorded; after the experiments are finished, the field in-situ test equipment is dismantled, and the removed variable-diameter hole inner bolt 4 or the bolt 3 having the variable-diameter hole is reinstalled at the upper cover plate 2 having the variable-diameter test hole to complete the indoor simulation experiments of the field in-situ tests.

After completing an indoor simulation experiment of field in-situ tests, the cooperative loading jack 7 is returned to an initial state through the loading console 14 based on a signal of the displacement sensor 13.

The upper fastening nut 1 is taken off, and the upper cover plate 2 having the variable-diameter test hole is removed.

The soil materials and the flexible sleeve 15 in the material barrel 6 having the measurement hole are moved out, sensors installed at the measurement hole of the material barrel 6 having the measurement hole are taken off, and the corresponding measurement hole bolt 10 is screwed on and tightened.

The upper cover plate 2 having the variable-diameter test hole is installed by aligning with the support column 5 according to the hole position, and the upper fastening nut 1 is tightened to complete shutdown.

The present application develops a large-scale indoor simulation device for in-situ soil layers and proposed a corresponding experiment method. On a premise of restoring a state of field in-situ soils, variable-diameter test holes and a plurality of measurement holes are provided, providing basic conditions for further conducting indoor simulation experiments of various field tests. Indoor simulation experiments of field in-situ tests that may be conducted by the device include: a cone penetration experiment, a dynamic penetration experiment, a standard penetration experiment, a vane shear experiment, a pressuremeter experiment, a shear wave velocity experiment, and the like. According to sizes of experiment soil materials and arrangement modes of sensors required for indoor simulation experiments of field in-situ tests to be conducted, sensors are arranged at the measurement hole at a suitable position, a test hole of a suitable size is selected, field test equipment is erected above the test hole, and different types of indoor simulation experiments of field in-situ tests are conducted.

The above described are only preferred embodiments of the present application and are not used to limit the present application. For those skilled in the art, the present application may have various modifications and changes. Any modification, equivalent replacement, improvement, and the like made within the scope of the present application shall fall within the protection scope of the present application.

What is claimed is:

1. A large-scale indoor simulation device for in-situ soil layers, comprising a base bottom plate, wherein
the base bottom plate is connected, through a cooperative loading jack, to a material barrel having a measurement hole, an inner wall of the material barrel is provided with a flexible sleeve, and an open end of the material barrel is provided with an upper cover plate having the variable-diameter test hole; a support column is utilized to penetrate the upper cover plate and the base bottom plate and is fixed by a nut; and
the cooperative loading jack is internally provided with a force sensor, an outer side of the cooperative loading jack is provided with a displacement sensor, and the cooperative loading jack, the force sensor, and the displacement sensor are all connected to a loading console.

2. The large-scale indoor simulation device of claim 1, wherein the support column is provided with a directional bearing, the directional bearing comprises a bearing bracket and a bearing, the support column passes through the bearing bracket, the directional bearing is fastened to the support column by utilizing the bearing bracket, and the bearing is in vertical contact with an outer side wall of the material barrel.

3. The large-scale indoor simulation device of claim 1, wherein a bolt having the variable-diameter hole is provided in the upper cover plate, a variable-diameter hole inner bolt is provided in the bolt having the variable-diameter hole, and a center of the variable-diameter hole inner bolt, a center of the bolt having the variable-diameter hole, and a center of the material barrel are coaxial.

4. The large-scale indoor simulation device of claim 1, wherein a positioning nut is utilized to fix between the support column and the upper cover plate, and a fastening nut is utilized to fix between the support column and the base bottom plate.

5. The large-scale indoor simulation device of claim 1, wherein a hole is provided at the flexible sleeve at a position corresponding to the measurement hole of the material barrel.

6. The large-scale indoor simulation device of claim 1, wherein a plurality sets of measurement hole bolts are provided at a side wall of the material barrel.

7. The large-scale indoor simulation device of claim 1, wherein a lubricating material is applied between the flexible sleeve and the material barrel.

8. A large-scale indoor simulation method for in-situ soil layers, applied to the large-scale indoor simulation device of claim 1, comprising:
placing a thin-layer flexible sleeve having an outer wall coated with a lubricating material into the material barrel, to closely fit with the inner wall of the material barrel; providing a hole at a position of the flexible sleeve corresponding to a position of the measurement hole of the material barrel;
loading experiment soil materials in layers into an interior of the material barrel having the measurement hole according to a preset relative density, and performing compaction for compactness restoration by utilizing a compactor after each layer is loaded; after a round of compaction, measuring and comparing whether the experiment soil materials are compacted to a designated height, and if not, performing a next round of compaction until the designated height is reached to complete loading and compaction of a layer of the experiment soil materials; during the compaction, removing a corresponding measurement hole bolt and arranging a sensor at a required position according to an experiment requirement;
installing the upper cover plate, starting the cooperative loading jack through the loading console, and loading the experiment soil materials to a preset stress state according to a signal of the force sensor to complete loading; and
conducting an indoor simulation experiment of a field in-situ test.

9. The large-scale indoor simulation method of claim 8, wherein the compaction separately adopts manual compaction and electric compaction according to a soil sample density requirement; during the manual compaction or the electric compaction, the compaction follows a basic principle of compacting from outside to inside, tamping a circle along the inner wall of the material barrel having the measurement hole, and moving the compactor inward to perform a next circle of compaction along an inner diameter of a compaction trajectory; during the compaction, two compaction trajectories partially overlap to ensure that a compaction range completely covers the experiment soil materials.

10. The large-scale indoor simulation method of claim 8, wherein a variable-diameter hole inner bolt or a bolt having the variable-diameter hole is removed before conducting the indoor simulation experiment.
